(19) United States
(12) Patent Application Publication
(10) Pub. No.: US 2003/0171617 A1
(43) Pub. Date: Sep. 11, 2003

(54) 3,4-Dihydroxymandelic acid alkylamides and use thereof

(76) Inventor: Jakob Peter Ley, Holzminden (DE)

Correspondence Address: BAYER POLYMERS LLC, 100 BAYER ROAD, PITTSBURGH, PA, 15205, US

(21) Appl. No.: 10/312,150

(22) PCT Filed: Jun. 11, 2001
(86) PCT No.: PCT/EP01/06567

(30) Foreign Application Priority Data
Jun. 23, 2000 (DE) 100 30 880.5

(51) Int. Cl. A61K 31/165; C07C 235/44

(52) U.S. Cl. 564/170; 514/622; 424/439

(57) ABSTRACT

The invention relates to N-alkyl-2-(3,4-dihydroxyphenyl)-2-hydroxyacetamides and to cosmetic and/or pharmaceutical preparations and foods comprising these compounds.

The invention relates to N-alkyl-2-(3,4-dihydroxyphenyl)-2-hydroxyacetamides, referred to below as 3,4-dihydroxymandelic acid alkylamides, and to cosmetic and/or pharmaceutical preparations and foods comprising these compounds.

For cosmetic and/or dermatological preparations, active ingredients are sought which, in physiological systems, in particular in or on the skin, the nails or hair of humans and animals, aid the natural defense mechanisms against free radicals and reactive oxygen compounds or, as protective substances in cosmetics, pharmaceuticals or foods, protect the oxidation-sensitive constituents thereof against autoxidation.

Antioxidants (oxidation inhibitors) are usually organic compounds which inhibit or prevent the undesired changes in the substances to be protected caused by oxygen effects including oxidative processes (Römpp Lexikon Chemie 10th edition, 229 (1996)). Many antioxidants also function as free-radical scavengers and/or as complexing agents for heavy metal ions.

The object of the present invention is to provide antioxidants with strong specific free-radical scavenging and/or antioxidative effect for use in cosmetic and pharmaceutical preparations and also for the protection of cells and tissue of humans and animals.

We have now found novel 3,4-dihydroxymandelic acid alkylamides of the general formula

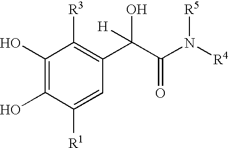

where

R¹, R² and R³, independently of one another, are hydrogen, lower alkyl or groups —O—R⁶ in which R⁶ is hydrogen or lower alkyl, and

R⁴ is hydrogen, an alkyl radical having 1 to 22 carbon atoms or an alkenyl radical having 2 to 22 carbon atoms, and

R⁵ is an alkyl radical having 1 to 22 carbon atoms or an alkenyl radical having 2 to 22 carbon atoms,

including stereoisomers thereof or mixtures thereof.

Surprisingly, the 3,4-dihydroxymandelic acid alkylamides according to the invention are very good free-radical scavengers and particularly strong antioxidants.

Preferably, the 3,4-dihydroxymandelic acid alkylamides according to the invention are, on the basis of their amphiphilic structure, suitable as antioxidants for highly unsaturated lipids and/or for multiphase mixtures of such lipids, e.g. with water. In particular, the 3,4-dihydroxymandelic acid alkylamides according to the invention are able to suppress the harmful effects of free radicals and/or oxidative processes which are induced by UV light on and/or in the human skin and to support the natural antioxidative processes. Additionally, it is advantageous that the 3,4-dihydroxymandelic acid alkylamides according to the invention do not hydrolyze in aqueous solutions or water-containing preparations, in particular at a pH between 4 and 10, to give the free acids.

Lower alkyl is generally a short-chain saturated, straight-chain, cyclic or branched hydrocarbon radical having, preferably, 1 to 4 carbon atoms. Specifically, mention may be made of: methyl, ethyl, n-propyl, isopropyl, cyclopropyl, n-butyl, sec-butyl, isobutyl, tert-butyl, cyclopropylmethyl or the various isomers of the methylcyclopropyl radical. Particular preference is given to methyl and ethyl.

Alkyl having 1 to 22 carbon atoms is generally a saturated, straight-chain, cyclic or branched hydrocarbon radical. The radical preferably contains 1 to 18, particularly preferably 1 to 12, carbon atoms. Specifically, mention may be made of: methyl, ethyl, n-propyl, isopropyl, n-butyl, sec-butyl, isobutyl, tert-butyl, the respective various straight-chain or branched isomers of the pentyl, hexyl, heptyl, octyl, nonyl, decyl, undecyl and dodecyl radical, cyclopentyl, cyclopentylmethyl, cyclopentylethyl, cyclopentylpropyl, the various isomers of the methylcyclopentyl radical, cyclohexyl, cycloheptyl, cyclooctyl, menthyl, isomenthyl, homomenthyl, norbornyl, bornyl, lauryl, myristyl, cetyl, isocetyl, stearyl and isostearyl. Particular preference is given to methyl, ethyl, n-propyl, isopropyl, cyclopropyl, n-butyl, sec-butyl, isobutyl, tert-butyl, cyclopentyl, cyclohexyl, menthyl, n-pentyl, n-hexyl, n-heptyl, n-octyl, n-nonyl, n-decyl, n-undecyl and lauryl.

Alkenyl having 2 to 22 carbon atoms is generally an unsaturated straight-chain, cyclic or branched hydrocarbon radical. The radical preferably contains 2 to 20, particularly preferably 2 to 12, carbon atoms. Specifically, mention may be made of: ethenyl, 1- or 2-propenyl, 1-, 2- or 3-butenyl, 2-methyl-1-propenyl, 2-methyl-2-propenyl, 1,3-butadienyl, 1,3-pentadienyl, 1,4-pentenyl, 2,4-pentenyl, the respective various straight-chain, cyclic or branched isomers of the pentenyl, hexenyl, heptenyl, octenyl, nonenyl and decenyl radical, and of the oleyl, linolyl, linolenyl, arachidyl and elaidyl radical. Particular preference is given to ethenyl, 1- or 2-propenyl, 1-, 2- or 3-butenyl, 2-methyl-1-propenyl, 2-methyl-2-propenyl, 3-methyl-1-pentenyl, 3-methyl-2-pentenyl, 3-methyl-3-pentenyl, cyclopentenyl, cyclohexenyl, pinenyl, norbornenyl and bornenyl.

Preference is given to the 3,4-dihydroxymandelic acid alkylamides of the formula

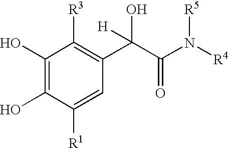

where

R¹, R² and R³, independently of one another, are hydrogen, methyl, tert-butyl, hydroxyl or methoxy, and

R⁴ is hydrogen, and

R⁵ is an alkyl radical having 1 to 18 carbon atoms or an alkenyl radical having 2 to 20 carbon atoms,

including stereoisomers thereof or mixtures thereof.

Particular preference is given to the 3,4-dihydroxymandelic acid alkylamides of the formula

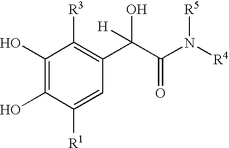

where

R¹, R², R³ and R⁴ are hydrogen, and

R⁵ is an alkyl radical having 1 to 12 carbon atoms or an alkenyl radical having 2 to 12 carbon atoms,

including stereoisomers thereof or mixtures thereof.

Individual compounds which may be mentioned are, for example, 2-(3,4-dihydroxyphenyl)-N-n-hexyl-2-hydroxyacetamide, N-cyclohexyl-2-(3,4-dihydroxyphenyl)-2-hydroxyacetamide and 2-(3,4-dihydroxyphenyl)-N-(2-ethylhexyl)-2-hydroxyacetamide.

The 3,4-dihydroxymandelic acid alkylamides according to the invention aid the natural defense mechanisms against free radicals and reactive oxygen compounds in physiological systems of the skin, the hair or the nails, and, in cosmetics, pharmaceuticals or foods, protect the oxidation-sensitive constituents thereof against autoxidation or photooxidation.

The 3,4-dihydroxymandelic acid alkylamides according to the invention can preferably be used in cosmetic or pharmaceutical preparations, preferably for protecting cells and tissues of mammals, in particular the skin of humans, and also in foods against the harmful effect of free radicals and reactive oxygen species. The preparations according to the invention can of course also be used analogously in other fields of use.

The amount of 3,4-dihydroxymandelic acid alkylamides in the cosmetic or pharmaceutical preparations according to the invention is 0.001% by weight to 30% by weight, preferably 0.001 to 20% by weight, particularly preferably 0.01% by weight to 5% by weight, based on the total weight of the preparation.

3,4-Dihydroxymandelic acid alkylamides for the purposes of the invention have hitherto not been described. Helvetica Chimica Acta 1963, pages 2271 et seq. describe the synthesis of 3,4-dihydroxymandelic acid amide by hydrolysis and subsequent cleavage of the benzyl groups of 2-(3,4-dibenzyloxyphenyl)-2-hydroxyacetonitrile. However, this process is not suitable in principle for the synthesis of the 3,4-dihydroxymandelic acid alkylamides according to the invention.

The 3,4-dihydroxymandelic acid alkylamides according to the invention can, however, be prepared using amide synthesis processes known per se by reacting an activated 3,4-dihydroxymandelic acid optionally protected on the OH groups with an alkylamine of the general formula HNR⁴R⁵ or an ammonium salt of the general formula (H₂NR⁴R⁵)⁺A⁻, where the radicals R⁴ and R⁵ have the meanings given above and A⁻ is an inorganic or organic anion, for example halide, sulfate, hydrogensulfate or acetate, optionally in the presence of solvents and auxiliary bases. Activated acid derivatives which may be used are the acid chlorides, the acid anhydrides or acid esters of, for example, optionally substituted phenols, N-hydroxysuccinimide or N-hydroxybenzotriazole. The protective groups used are preferably acyl, carbamate or ether groups, e.g. acetyl, benzoyl, methoxycarbonyl, tert-butoxycarbonyl, allyl or benzyl groups. Solvents which may be used are, for example, water, acetone, 1,4-dioxane, N,N-dimethylformamide, tetrahydrofuran, ethyl acetate, chloroform or else mixtures of the last-named solvents. Auxiliary bases which may be used are, for example, ammonium, alkali metal or alkaline earth metal carbonates, hydrogencarbonates, hydroxides, tertiary amines and inorganic or organic basic ion exchangers.

The 3,4-dihydroxymandelic acid alkylamides according to the invention are particularly preferably prepared from 3,4-dihydroxymandelic acid N-succinimidyl esters optionally blocked on the hydroxyl groups with acetyl or methoxycarbonyl groups with alkylamines or ammonium salts thereof in a water-containing solvent mixture, preferably a water/1,4-dioxane or water/acetone mixture with one of the abovementioned auxiliary bases at 5 to 100° C. Advantageously, the 3,4-dihydroxymandelic acid N-succinimidyl esters optionally blocked on the hydroxyl groups with acetyl or methoxycarbonyl groups are synthesized from the corresponding free acid and N-hydroxysuccinimide (NHOSu) by means of a carbodiimide, preferably N,N′-dicyclohexylcarbodiimide (DCC), in an aprotic solvent, preferably 1,4-dioxane, diethyl ether, tert-butyl methyl ether, ethyl acetate or tetrahydrofuran, at 0 to 50° C., preferably at 5 to 30° C., the dissolved crude product is separated from the residue by filtration, and the filtrate is reacted directly within the meaning of the invention with the initial charge of alkylamine or ammonium salt thereof or one of the abovementioned auxiliary bases in water or water/1,4-dioxane or water/acetone mixture. The process is illustrated by the following scheme using 2-(3,4-dihydroxyphenyl)-N-(2-ethylhexyl)-2-hydroxyacetamide as an example:

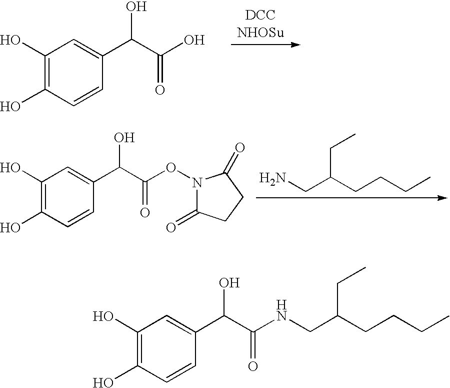

The 3,4-dihydroxymandelic acids used are, in particular, 2-(3,4-dihydroxyphenyl)-2-hydroxyacetic acid (3,4-dihydroxymandelic acid), and stereoisomers or mixtures thereof.

The alkylamines used are, in particular, n-hexylamine, 2-ethylhexylamine or cyclohexylamine or their corresponding ammonium salts.

The 3,4-dihydroxymandelic acid alkylamides according to the invention can, however, also be obtained by direct condensation of the free acids with an alkylamine of the general formula HNR⁴R⁵, where the radicals R⁴ and R⁵ have the meanings given above, with or without solvents. The reaction is illustrated in the scheme below using 2-(3,4-dihydroxyphenyl)-N-hexyl-2-hydroxyacetamide as an example:

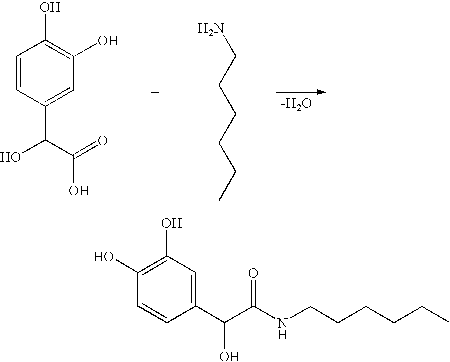

Condensing agents which may be used are, for example, carbodiimides, preferably N,N′-dicyclohexylcarbodiimide, and solvents which may be used are, for example, 1,4-dioxane, diethyl ether, tert-butyl methyl ether, ethyl acetate or tetrahydrofuran.

The 3,4-dihydroxymandelic acid alkylamides according to the invention are obtained from these reaction mixtures by purification steps known per se; where appropriate, any protective groups still present have to be cleaved off using methods known per se.

The cosmetic and pharmaceutical preparations according to the invention comprise the 3,4-dihydroxymandelic acid alkylamides in an effective amount, alongside other, otherwise customary composition constituents. They comprise 0.001% by weight to 30% by weight, preferably 0.001 to 20% by weight, in particular 0.01% by weight to 5% by weight, based on the total weight of the formulation, of the 3,4-dihydroxymandelic acid alkylamides according to the invention and can be in the form of “water-in-oil”, “oil-in-water”, “water-in-oil-in-water” or “oil-in-water-in-oil” emulsions, microemulsions, gels, solutions, e.g. in oils, alcohols or silicone oils, sticks, soaps, aerosols, sprays and also foams. Further customary cosmetic auxiliaries and additives may be present in amounts of 5-99.999% by weight, preferably 10-80% by weight, based on the total weight of the formulation. In addition, the formulations can have water in an amount up to 99.999% by weight, preferably 5-80% by weight, based on the total weight of the formulation.

The cosmetic or pharmaceutical preparations according to the invention are prepared by customary processes well known to the person skilled in the art by incorporating one or more of the 3,4-dihydroxymandelic acid alkylamides into cosmetic or pharmaceutical preparations which have the customary composition and preferably can be used for the treatment, the protection, the care and the cleansing of the skin, the nails or the hair and as make-up products in decorative cosmetics.

The foods or luxury products according to the invention, particularly preferably preparations for nutrition or food supplementing, comprise the 3,4-dihydroxymandelic acid alkylamides according to the invention in an effective amount, alongside other, otherwise customary composition constituents. They comprise 0.001 to 5% by weight, preferably 0.001 to 1% by weight, but in particular 0.01 to 0.5% by weight, based on the total weight of the preparation, of the 3,4-dihydroxymandelic acid alkylamides according to the invention and may be in the form, for example, of solids, pastes, emulsions, dispersions and also drinkable liquid preparations. Further customary food constituents, e.g. fats, oils, plant constituents, animal constituents, low and high molecular weight carbohydrates, proteins, peptides, amino acids, spices, sweeteners, inorganic and organic salts, flavor modifiers, fragrances and flavors, thickeners, preservatives, emulsifiers and dyes can be present in amounts of from 0.0001 to 99.999% by weight, preferably 1 to 90% by weight, based on the total weight of the preparation. In addition, the formulations can have water in an amount up to 99.999% by weight, based on the total weight of the preparation.

The foods or luxury products according to the invention are prepared using the customary processes well known to the person skilled in the art by incorporating one or more of the 3,4-dihydroxymandelic acid alkylamides according to the invention into preparations for nutrition or food supplement which have the customary composition and are suitable with regard to nutrition as luxury product and/or as food supplement with antioxidative effect for humans and for animals.

To prepare the cosmetic and pharmaceutical preparations or the foods or luxury products according to the invention, in a further embodiment, the 3,4-dihydroxymandelic acid alkylamides according to the invention are also incorporated beforehand into liposomes, e.g. starting from phosphatidylcholine, into microspheres, into nanospheres or else into capsules made of a suitable matrix, e.g. made of natural or synthetic waxes, for example beeswax, carnauba wax, silicone wax or paraffin waxes, and also stearyl alcohol, eicosanol, cetyl alcohol, stearin or made of gelatin.

For use, the cosmetic and dermatological preparations according to the invention are applied to the skin, the nails and/or the hair in an adequate amount in the manner customary for cosmetics.

The cosmetic and dermatological preparations according to the invention can comprise cosmetic auxiliaries and additives as are customarily used in such preparations, e.g. sunscreens (e.g. organic or inorganic light filter substances, preferably micropigments), preservatives, bactericides, fungicides, virucides, cooling active ingredients, plant extracts, antiinflammatory active ingredients, substances which accelerate wound healing (e.g. chitin or chitosan and derivatives thereof), film-forming substances (e.g. polyvinylpyrrolidones or chitosan or derivatives thereof), customary antioxidants, vitamins (e.g. vitamin C and derivatives, tocopherols and derivatives, vitamin A and derivatives), 2-hydroxycarboxylic acids (e.g. citric acid, malic acid, L-, D-, or dl-lactic acid), skin-lightening agents (e.g. kojic acid, hydroquinone or arbutin), skin-coloring agents (e.g. walnut extracts or dihydroxyacetone), perfumes, antifoams, dyes, pigments which have a coloring action, thickeners, surface-active substances, emulsifiers, emollients, moisturizers and/or humectants (e.g. glycerol or urea), fats, oils, unsaturated fatty acids or derivatives thereof (e.g. linoleic acid, -linolenic acid, γ-linolenic acid or arachidonic acid and natural or synthetic esters thereof in each case), waxes or other customary constituents of a cosmetic or dermatological formulation, such as alcohols, polyols, polymers, foam stabilizers, electrolytes, organic solvents, silicone derivatives or chelating agents (e.g. ethylenediaminetetraacetic acid and derivatives).

The amounts of cosmetic or dermatological auxiliaries and additives and perfume to be used in each case can be readily determined by the person skilled in the art by simple experimentation depending on the nature of the product in question.

Preferably, the preparations according to the invention can also additionally comprise one or more of the 3,4-dihydroxymandelic acid alkylamides according to the invention and also one or more other antioxidants. The antioxidants are advantageously chosen from the group consisting of amino acids (e.g. glycine, histidine, 3,4-dihydroxyphenylalanine, tyrosine, tryptophan) and derivatives thereof, imidazoles (e.g. urocaninic acid) and derivatives thereof, peptides (D,L-carnosine, D-carnosine, L-carnosine, anserine) and derivatives thereof, carotenoids, carotenes (e.g. α-carotene, β-carotene, lycopene) and derivatives thereof, chlorogenic acid and derivatives thereof, lipoic acid and derivatives thereof, aurothioglucose, propylthiouracil and other thiols (e.g. thioredoxin, glutathione, cysteine, cystine, cystamine and the glycosyl and N-acyl derivatives thereof or alkyl esters thereof), and salts thereof, dilauryl thiodipropionate, distearyl thiodipropionate, thiodipropionic acid and derivatives thereof, and phenolic acid amides of phenolic benzylamines (e.g. homovanillic acid amides, 3,4-dihydroxyphenylacetic acid amides, ferulic acid amides, sinapic acid amides, caffeic acid amides, dihydroferulic acid amides, dihydrocaffeic acid amides, vanillomandelic acid amides or 3,4-dihydroxymandelic acid amides of 3,4-dihydroxybenzylamine, 2,3,4-trihydroxybenzylamine or 3,4,5-trihydroxybenzylamine), catechol oximes (e.g. 3,4-dihydroxybenzaldoxime or 3,4-dihydroxybenzaldehyde O-ethyloxime), and also (metal) chelating agents (e.g. 2-hydroxy fatty acids, phytic acid, lactoferrin), humic acid, bile acids, bile extracts, bilirubin, biliverdin, folic acid and derivatives thereof, ubiquinone and ubiquinol and derivatives thereof, vitamin C and derivatives thereof (e.g. ascorbyl palmitate, magnesium ascorbyl phosphate, ascorbyl acetate), tocopherols and derivatives (e.g. vitamin E acetate), vitamin A and derivates (e.g. vitamin A palmitate), rutinic acid and derivatives thereof, flavonoids (e.g. quercetin, α-glucosylrutin) and derivatives thereof, phenolic acids (e.g. gallic acid, ferulic acid) and derivatives thereof (e.g. propyl gallate, ethyl gallate, octyl gallate), furfurylideneglucitol, dibutylhydroxytoluene, butylhydroxyanisole, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, resveratrol) and the derivatives of these said active ingredients which are suitable according to the invention.

The amount of further antioxidants in the preparations according to the invention can generally be 0.001 to 30% by weight, preferably 0.001 to 20% by weight, particularly preferably 0.001 to 5% by weight, based on the total weight of the preparation.

Apart from the 3,4-dihydroxymandelic acid alkylamides according to the invention, two or more further antioxidants can of course be used.

In the cosmetic or pharmaceutical preparations according to the invention, however, it is also possible to use UV-A and/or UV-B filter substances, where the total amount of filter substances may be 0.1 to 30% by weight, preferably 0.5 to 10% by weight, based on the total weight of the preparations, giving, for example, sunscreens for skin and hair. UV-A and/or UV-B filter substances which may be used are, for example, 3-benzylidenecamphor derivatives (e.g. 3-(4-methylbenzylidene)-dl-camphor), aminobenzoic acid derivatives (e.g. 2-ethylhexyl 4-(N,N-dimethylamino)benzoate or menthyl anthranilate), 4-methoxycinnamates (e.g. 2-ethylhexyl p-methoxycinnamate or isoamyl p-methoxycinnamate), benzophenones (e.g. 2-hydroxy-4-methoxybenzophenone), mono- or polysulfonated UV filters [e.g. 2-phenylbenzimidazole-5-sulfonic acid, sulisobenzones or 1,4-bis-(benzimidazolyl)benzene-4,4′,6,6′-tetrasulfonic acid and 3,3′-(1,4-phenylenedimethylidene)bis(7,7-dimethyl-2-oxobicyclo[2,2,1]heptane-1-methanesulfonic acid) and salts thereof], salicylates (e.g. 2-ethylhexyl salicylate or homomenthyl salicylate), triazines {e.g. 2,4-bis[4-(2-ethylhexyloxy)-2-hydroxyphenyl]-6-(4-methoxyphenyl)-1,3,5-triazine, bis(2-ethylhexyl) 4,4′-([6-([(1,1-dimethylethyl)aminocarbonyl]phenylamino)-1,3,5-triazine-2,4-diyl]diimino)bisbenzoate)}, 2-cyanopropenoic acid derivatives (e.g. 2-ethylhexyl 2-cyano-3,3-diphenyl-2-propenoate), dibenzoyl derivatives (e.g. 4-tert-butyl-4′-methoxydibenzoylmethane), polymer-bonded UV filters (e.g. polymers of N-[2-(or 4)-(2-oxo-3-bornylidene)methyl]benzylacrylamide) or pigments (e.g. titanium dioxides, zirconium dioxides, iron oxides, silicon dioxides, manganese oxides, aluminum oxides, cerium oxides or zinc oxides).

The lipid phase in the cosmetic and/or pharmaceutical preparations according to the invention can advantageously be chosen from the following groups of substances: mineral oils (advantageously paraffin oil), mineral waxes, hydrocarbons (advantageously squalane or squalene), synthetic or semisynthetic triglyceride oils (e.g. triglycerides of capric or caprylic acid), natural oils (e.g. castor oil, olive oil, sunflower oil, soybean oil, peanut oil, rapeseed oil, almond oil, palm oil, coconut oil, palm kernel oil, borage seed oil and the like), natural ester oils (e.g. jojoba oil), synthetic ester oils (preferably esters of saturated and/or unsaturated, linear and/or branched alkanecarboxylic acids having 3 to 30 carbon atoms with saturated and/or unsaturated, linear and/or branched alcohols having 3 to 30 carbon atoms and esters of aromatic carboxylic acids with saturated and/or unsaturated, linear and/or branched alcohols having 3 to 30 carbon atoms, in particular chosen from the group consisting of isopropyl myristate, isopropyl stearate, isopropyl palmitate, isopropyl oleate, n-butyl stearate, n-hexyl laurate, n-decyl laurate, isooctyl stearate, isononyl stearate, isononyl isononanoate, 2-ethylhexyl palmitate, 2-ethylhexyl laureate, 2-hexyldecyl stearate, 2-octyldecyl palmitate, oleyl oleate, oleyl erucate, erucyl oleate, erucyl erucate, and synthetic or natural mixtures of such esters), fats, waxes and other natural and synthetic fatty bodies, preferably esters of fatty alcohols with alcohols of low carbon number (e.g. with isopropanol, propylene glycol or glycerol) or esters of fatty alcohols with alkanoic acids of low carbon number or with fatty acids, alkyl benzoates (e.g. mixtures of n-dodecyl, n-tridecyl, n-tetradecyl and n-pentadecyl benzoate), and cyclic or linear silicone oils (such as, for example, dimethylpolysiloxanes, diethylpolysiloxanes, diphenylpolysiloxanes and mixed forms thereof).

The aqueous phase of the cosmetic and pharmaceutical preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl ether, monoethyl or monobutyl ether, diethylene glycol monomethyl or monoethyl ether and analogous products, and also alcohols of low carbon number, e.g. ethanol, isopropanol, 1,2-propanediol, glycerol, and also α- or β-hydroxy acids, preferably lactic acid, citric acid or salicylic acid, and also emulsifiers which can advantageously be chosen from the group of ionic, nonionic, polymeric, phosphate-containing and zwitterionic emulsifiers, and in particular one or more thickeners which can advantageously be chosen from the group consisting of silicon dioxide, aluminum silicates, such as, for example, bentonites, polysaccharides and derivatives thereof, e.g. hyaluronic acid, guar kernal flour, xanthan gum, hydroxypropylmethylcellulose or allulose derivatives, particularly advantageously from the group of polyacrylates, preferably a polyacrylate from the group of carbopols, in each case individually or in combination or from the group of polyurethanes.

Particular preference is given to the use of the cosmetic or pharmaceutical preparations according to the invention for protecting tissues and cells of mammals, in particular of the skin, the hair and/or the nails of humans, against oxidative stress and the harmful effect of free radicals.

The present invention likewise also covers a process for protecting cosmetic or pharmaceutical preparations and foods and luxury products against oxidation or photooxidation, these preparations being, for example, preparations for the treatment, protection and care of the skin, the nails or of the hair and also make-up products, constituents of which encounter stability problems due to oxidation or photooxidation during storage, characterized in that the cosmetic or pharmaceutical preparations, and the foods or luxury products have an effective content of 3,4-dihydroxymandelic acid alkylamides according to the invention.

### EXAMPLES

### Example 1

### 2-(3,4-Dihydroxyphenyl)-N-(2-ethylhexyl)-2-hydroxyacetamide

3,4-Dihydroxymandelic acid (500 mg, 2.72 mmol) was initially introduced together with N-hydroxysuccinimide (313 mg, 2.72 mmol) and N,N′-dicyclohexylcarbodiimide (560 mg, 2.717 mmol) in dry 1,4-dioxane (30 ml) and stirred under nitrogen for 16 h at room temperature. After the precipitate had been filtered off, the filtrate was added to a preprepared solution of 2-ethylhexylamine (376 mg, 3.26 mmol) and sodium hydrogencarbonate (271 mg, 3.26 mmol) in 1,4-dioxane/water (1:1, 20 ml), and the reaction mixture was stirred for a further 2.5 h at 50° C. in a water bath and left to cool. The mixture is adjusted to be acidic with hydrochloric acid, extracted 3 times with ethyl acetate, and the organic phase is washed with saturated aqueous sodium chloride solution, dried over sodium sulfate and filtered, and the filtrate is concentrated by evaporation at 40° C./160 mbar. The crude product is chromatographed over silica gel 60 with ethyl acetate. Yield: 567 mg (75%, 98% according to HPLC). ¹H-NMR (200 MHz, d₆-DMSO): δ=7.68 (1H, t, 6 Hz), 7.77 (1H, m), 7.61 (2H, m), 4.67 (1H, s), 2.97 (dd), 1.80-1.05 (9 H, m), 0.90-0.70 (6H, m) ppm. MS (APCI neg.) m/e=295.25 (100%, [M−H]⁻), 277.87 (35%).

The following compounds were prepared according to an analogous procedure:

### Example 2

### N-Cyclohexyl-2-(3,4-dihydroxyphenyl)-2-hydroxyacetamide

¹H-NMR (200 MHz, d₆-DMSO): δ=8.83 (2H, bs), 7.55 (1H, d, 8 Hz), 6.76 (1H, m), 6.62 (2H, m), 5.83 (1H, bs), 4.66 (1H, s), 3.60-3.31 (m), 1.82-1.50 (m), 1.50-1.00 (m) ppm. MS (APCI neg.) m/e=264.66 (100%, [M−H]⁻).

### Example 3

### 2-(3,4-Dihydroxyphenyl)-N-n-hexyl-2-hydroxyacetamide

¹H-NMR (400 MHz, CD₃OD): δ=6.85 (1H, m), 6.75-6.69 (2H, m), 4.85 (partially suppressed, s), 3.20 (2H, t, 7 Hz), 1.56-1.45 (2H, m), 1.40-1.25 (6H, m), 0.90 (3H, t, 7 Hz) ppm. MS (APCI pos.) m/e=267.84 (100%, [M+H]⁺), 249.92 (80%), 222.20 (35%), 545.67 (21%, [2M+H]⁺).

### Preparation of the Preparations

### Example 4

### Cosmetic Solution Containing 2-(3,4-dihydroxyphenyl)-N-n-hexyl-2-hydroxyacetamide

| | Content |
|---|---|
| | in % by |
| Raw material name | wt. |
| 1,3-Butylene glycol | 99.9 |
| 2-(3,4-Dihydroxyphenyl)-N-n-hexyl-2-hydroxyacetamide | 0.1 |

### Example 5

### “Oil-in-Water” Emulsion Containing 2-(3,4-dihydroxyphenyl)-N-n-hexyl-2-hydroxyacetamide

| | Raw material | | Content |
|---|---|---|---|
| | name | | in % by |
| Part | (manufacturer) | Chemical name | wt. |
| A | Arlatone 983 S ® | Ether of polyethylene glycol | 1.2 |
| | (ICI) | with glyceryl monostearate | |
| | Brij 76 ® (ICI) | 3,6,9,12,15,18,21,24,27,30,33, | 1.2 |
| | | 36-Decaoxaoctatetracontan-1- | |
| | | ol | |
| | Cutina MD ® | Glyceryl monostearate | 3.5 |
| | (Henkel) | | |
| | Baysilone oil | Polydimethylsiloxane | 0.8 |
| | M10 ® | | |
| | (GE Bayer) | | |
| | Eutanol G ® | Octyldodecanol | 3.0 |
| | (Henkel) | | |
| | 2-(3,4-Dihy- | | 0.1 |
| | droxyphenyl)- | | |
| | N-n-hexyl-2-hy- | | |
| | droxyacetamide | | |
| | Paraffin oil 65 cp | Mineral oil | 8.0 |
| | (Henry Lamotte) | | |
| B | Water, dist. | | 49.8 |
| | | 2-Phenoxyethanol and methyl | |
| | | 4-hydroxybenzoate and ethyl | |
| | Phenopip ® | 4-hydroxybenzoate and propyl | 0.5 |
| | (Nipa | 4-hydroxybenzoate and butyl | |
| | Laboratories) | 4-hydroxybenzoate | |
| | 1,2-propylene | | 2.0 |
| | glycol | | |
| | Glycerol 99% | | 3.0 |
| C | Water, dist. | | 25.0 |
| | Carbopol 2050 ® | Crosslinked acrylic acid/ | 0.4 |
| | (B. F. Goodrich) | C₁₀-C₃₀-alkyl acrylate polymer | |
| | Aqueous sodium | | 1.2 |
| | hydroxide solu- | | |
| | tion, 10% | | |
| D | Perfume oil | | 0.3 |

Part A was mixed and heated to 80° C. Part B was mixed and heated to 90° C. and added to part A with stirring. For part C, Carbopol was carefully dispersed in water and neutralized with sodium hydroxide solution (pH 6.5). Part C was then added at 60° C. to the mixture of parts A and B. Part D was added to the mixture of parts A, B, and C at room temperature.

### Example 6

### “Oil-in-Water” Emulsion Containing 2-(3,4-dihydroxyphenyl)-N-(2-ethylhexyl)-2-hydroxyacetamide

| | Raw material | | Content |
|---|---|---|---|
| | name | | in % by |
| Part | (manufacturer) | Chemical name | wt. |
| A | Arlatone 983 S ® | Ether of polyethylene glycol | 1.2 |
| | (ICI) | with glyceryl monostearate | |
| | Brij 76 ® (ICI) | 3,6,9,12,15,18,21,24,27,30,33, | 1.2 |
| | | 36-Decaoxaoctatetracontan-1- | |
| | | ol | |
| | Cutina MD ® | Glyceryl monostearate | 3.5 |
| | (Henkel) | | |
| | Baysiloneoil | Polydimethylsiloxane | 0.8 |
| | M10 ® | | |
| | (GE Bayer) | | |
| | Eutanol G ® | Octyldodecanol | 3.0 |
| | (Henkel) | | |
| | 2-(3,4-Dihy- | | 0.2 |
| | droxyphenyl)-N- | | |
| | (2-ethylhexyl)-2- | | |
| | hydroxyacetamide | | |
| | Paraffin oil 65 cp | Mineral oil | 8.0 |
| | (Henry Lamotte) | | |
| B | Water, dist. | | 49.8 |
| | | 2-Phenoxyethanol and methyl | |
| | | 4-hydroxybenzoate and ethyl | |
| | Phenopip ® | 4-hydroxybenzoate and propyl | 0.5 |
| | (Nipa | 4-hydroxybenzoate and butyl | |
| | Laboratories) | 4-hydroxybenzoate | |
| | 1,2-propylene | | 2.0 |
| | glycol | | |
| | Glycerol 99% | | 3.0 |
| C | Water, dist. | | 25.0 |
| | Carbopol 2050 ® | Crosslinked acrylic acid/ | 0.4 |
| | (B. F. Goodrich) | C₁₀-C₃₀-alkyl acrylate polymer | |
| | Aqueous sodium | | 1.2 |
| | hydroxide solu- | | |
| | tion, 10% | | |
| D | Perfume oil | | 0.3 |

Part A was mixed and heated to 80° C. Part B was mixed and heated to 90° C. and added to part A with stirring. For part C, Carbopol was carefully dispersed in water and neutralized with sodium hydroxide solution (pH 6.5). Part C was then added at 60° C. to the mixture of parts A and B. Part D was added to the mixture of parts A, B, and C at room temperature.

### Example 7

### “Water-in-Oil” Sunscreen Emulsion with UVA/B Broadband Protection and 2-(3,4-dihydroxyphenyl)-N-(2-ethylhexyl)-2-hydroxyacetamide

| | | | Content |
|---|---|---|---|
| | Raw material name | | in % by |
| Part | (manufacturer) | Chemical name | wt. |
| A | Dehymuls PGPH ® | Polyglycerol-2 dipolyhydroxy | 3.0 |
| | (Henkel) | stearate | |
| | Monomuls 90-O 18 ® | Glyceryl oleate | 1.0 |
| | (Henkel) | | |
| | Permulgin 2550 ® | Beeswax | 1.0 |
| | (Koster Keunen | | |
| | Holland) | | |
| | Myritol 318 ® (Henkel) | Caprylic/capric triglycerides | 6.0 |
| | Witconol TN ® (Witco) | C₁₂-C₁₅-alkyl benzoate | 6.0 |
| | Cetiol SN ® (Henkel) | Cetyl and stearyl isononanoate | 5.0 |
| | Copherol 1250 ® | Tocopherol acetate | 1.0 |
| | (Henkel) | | |
| | Solbrol P ® (Bayer) | Propyl 4-hydroxybenzoate | 0.1 |
| | Neo Heliopan ® AV | 2-Ethylhexyl p-methoxy- | 4.0 |
| | (Haarmann & Reimer) | cinnamate | |
| | Neo Heliopan ® E 1000 | Isoamyl p-methoxycinnamate | 4.0 |
| | (Haarmann & Reimer) | | |
| | Neo Heliopan ® MBC | 3-(4-Methylbenzylidene)-dl- | 2.0 |
| | (Haarmann & Reimer) | camphor | |
| | Neo Heliopan ® OS | 2-Ethylhexyl salicylate | 3.0 |
| | (Haarmann & Reimer) | | |
| | Octyltriazone | | 1.0 |
| | 2-(3,4-Dihydroxy- | | 0.1 |
| | phenyl)-N-(2-ethyl- | | |
| | hexyl)-2-hydroxyaceta- | | |
| | mide | | |
| | Zinc oxide neutral | | 7.0 |
| | (Haarmann & Reimer) | | |
| B | Water, dist. | | 40 |
| | Phenoxyethanol | | 0.7 |
| | Solbrol M (Bayer) | Methyl 4-hydroxybenzoate | 0.2 |
| | Glycerol 99% | | 4.0 |
| | Neo Heliopan ® Hydro | 2-Phenylbenzimidazole-5- | 10.0 |
| | (Haarmann & Reimer), | sulfonic acid | |
| | 15% as sodium salt | | |
| | Benzophenone-4 | | 0.5 |
| C | Perfume oil | | 0.3 |
| | Bisabolol | | 0.1 |

For part A, all of the substances apart from the zinc oxide were heated to 85° C. and the zinc oxide was carefully dispersed in the mixture. The components of part B were mixed, heated to 85° C. and added to part A with stirring. Part C was added to the mixture of parts A and B and then the mixture was homogenized using a dispersion tool.

### Example 8

### “Oil-in-Water” Sunscreen Emulsion with UVA/B Broadband Protection and N-cyclohexyl-2-(3,4-dihydroxyphenyl)-2-hydroxyacetamide

| | | | Content |
|---|---|---|---|
| | Raw material name | | in % by |
| Part | (manufacturer) | Chemical name | wt. |
| A | Arlacel 165 ® (ICI) | Glyceryl stearate and | 3.0 |
| | | polyethylene glycol 100 | |
| | | stearate | |
| | Emulgin B2 ® (Henkel) | Ceteareth-20 | 1.0 |
| | Lanette O ® (Henkel) | Cetyl and stearyl alcohol | 1.15 |
| | Myritol 318 ® (Henkel) | Caprylic/capric triglycerides | 5.0 |
| | Cetiol SN ® (Henkel) | Cetyl and stearyl isononanoate | 4.0 |
| | Abil 100 ® | Polydimethylsiloxane | 1.0 |
| | (Goldschmidt) | | |
| | Bentone Gel MIO ® | Mineral oil and quatemium-18 | 3.0 |
| | (Rheox) | hectorite and propylene | |
| | | carbonate | |
| | Cutina CBS ® (Henkel) | Glyceryl stearate and cetyl | 2.0 |
| | | alcohol and stearyl alcohol | |
| | | and cetyl palmitate and coco | |
| | | glycerides | |
| | Neo Heliopan ® 303 | 2-Ethylhexyl-2-cyano-3,3- | 7.0 |
| | (Haarmann & Reimer) | diphenyl-2-propenoate | |
| | Neo Heliopan ® BB | 2-Hydroxy-4-methoxybenzo- | 1.0 |
| | (Haarmann & Reimer) | phenone | |
| | Neo Heliopan ® MA | Menthyl anthranilate | 3.0 |
| | (Haarmann & Reimer) | | |
| | 2-Ethylhexyl N,N-di- | | 3.0 |
| | methyl-4-aminobenzoate | | |
| | N-Cyclohexyl-2-(3,4- | | 0.1 |
| | dihydroxyphenyl)-2- | | |
| | hydroxyacetamide | | |
| | Titanium dioxide, | | 5.0 |
| | microfine | | |
| B | Water, dist. | | 55.85 |
| | Veegum ultra ® | Magnesium aluminum sulfate | 1.0 |
| | (Vanderbilt) | | |
| | Natrosol 250 HHR | Hydroxymethylcellulose | 0.3 |
| | (Hercules) | | |
| | Glycerol | | 3.0 |
| | | 2-Phenoxyethanol and methyl | |
| | | 4-hydroxybenzoate and ethyl | |
| | Phenopip ® (Nipa | 4-hydroxybenzoate and propyl | 0.3 |
| | Laboratories) | 4-hydroxybenzoate and butyl | |
| | | 4-hydroxybenzoate | |
| C | Perfume oil | | 0.3 |

For part A, all of the substances apart from the titanium dioxide were mixed and heated to 85° C.; the titanium dioxide was carefully dispersed into the mixture. For part B, all of the substances apart from Veegum and Natrosol were mixed, heated to 90° C., Natrosol and Veegum were dispersed therein and the mixture was added to part A with stirring. Part C was added to the mixture of parts A and B and then the mixture was homogenized using a dispersion tool (pH 5.6).

### Example 9

### “Oil-in-Water” Sunscreen Emulsion with UVA/B Broadband Protection and N-cyclohexyl-2-(3,4-dihydroxyphenyl)-2-hydroxyacetamide

| | | | Content |
|---|---|---|---|
| | Raw material name | | in % by |
| Part | (manufacturer) | Chemical name | wt. |
| A | Crodaphos MCA ® | Cetyl phosphate | 1.50 |
| | (Croda) | | |
| | Cutina MD ® (Henkel) | Glyceryl stearate | 2.0 |
| | Lanette 16 ® (Henkel) | Cetyl alcohol | 1.2 |
| | Myritol 318 ® (Henkel) | Caprylic/capric triglycerides | 5.0 |
| | Cetiol SN ® (Henkel) | Cetyl and stearyl isononanoate | 5.0 |
| | Copherol 1250 ® | Tocopherol acetate | 0.5 |
| | (Henkel) | | |
| | Solbrol P ® (Bayer) | Propyl 4-hydroxybenzoate | 0.1 |
| | Abil 100 ® | Polydimethyl siloxane | 0.3 |
| | (Goldschmidt) | | |
| | Neo Heliopan ® HMS | 3,3,5-Trimethylcyclohexyl | 5.0 |
| | (Haarmann & Reimer) | salicylate | |
| | N-Cyclohexyl-2-(3,4- | | 0.1 |
| | dihydroxyphenyl)-2- | | |
| | hydroxyacetamide | | |
| | Butylmethoxydibenzoyl- | | 2.0 |
| | methane | | |
| B | Water, dist. | | 47.8 |
| | 1,3-Butylene glycol | | 3.0 |
| | Sobrol M ® (Bayer) | Methyl 4-hydroxybenzoate | 0.2 |
| | Phenoxyethanol | | 0.7 |
| | Carbopol ETD 2050 ® | Copolymer acrylic acid/C₁₀- | 0.2 |
| | (B. F. Goodrich) | C₃₀-alkyl acrylate | |
| | Keltrol T ® (Calgon) | Xanthan gum | 0.2 |
| | Neo Heliopan ® AP | 2,2-(1,4-Phenylene)bis(1H- | 22 |
| | (Haarmann & Reimer) | benzimidazole-4,6-disulfonic | |
| | | acid) and disodium salt | |
| C | Aqueous sodium | | 2.8 |
| | hydroxide solution, 10% | | |
| D | Perfume oil | | 0.3 |
| | Bisabolol | | 0.1 |

Part A was heated to 85° C. Part B: Carbopol and Keltrol were dispersed into the remaining constituents while cold, the mixture was heated to 85° C. and added to part A. Part C was immediately added at 80° C. to the mixture of parts A and B and dispersed for 5 min using a dispersion tool. Finally, part D was added at room temperature and the mixture was homogenized using a dispersion tool (pH 6.6).

### Demonstration of Activity

### Example 10

### Activity as Free-Radical Scavengers

The activity of the exemplary compounds as in examples 1 to 3 as free-radical scavengers was compared with that of conventional free-radical scavengers. For this purpose, the DPPH (1,1-diphenyl-2-picrylhydrazyl) test for the removal of free radicals was used.

DPPH was dissolved in methanol to a concentration of 100 μmol/l. A series of dilutions of the exemplary compounds, vitamin C, α-tocopherol and dibutylhydroxytoluene were prepared in methanol. Methanol served as the control. 2500 μl of the DPPH solution were mixed with 500 μl of each test solution and the decrease in absorption at 515 nm was read until the decrease was less than 2% per hour. The activity of the test substances as free-radical scavengers was calculated according to the following equation:

Activity as free-radical scavenger (%)=100−(absorption of the test compounds)/(absorption of the control)×100.

The activity as free-radical scavenger (%) in a series of dilutions of test compounds was used to calculate, for each test compound, the effective relative concentration EC₅₀ (based on the starting concentration of DPPH, EC=c (test compound)/c(DPPH)) of a test compound at which 50% of the free radical DPPH had been removed. The results are given in table 1:

TABLE 1

| Test compound as in example | EC₅₀/(mol/mol) |
|---|---|
| 1 | 0.12 |
| 2 | 0.11 |
| 3 | 0.12 |
| Vitamin C | 0.27 |
| α-Tocopherol | 0.25 |
| Dibutylhydroxytoluene | 0.24 |

### Example 11

### Activity as Antioxidants

The activity of the exemplary compounds as in examples 1 to 3 as antioxidants was compared with that of conventional antioxidants. The test system used was the accelerated autoxidation of lipids by air with or without antioxidant using the Rancimat apparatus (Rancimat is a registered trademark of Metrohm AG, Herisau, Switzerland).

The exemplary compounds, vitamin C, α-tocopherol and dibutylhydroxytoluene were dissolved in methanol or acetone, and 100 μl of each test solution were added to a 3 g preprepared oil sample. In a control sample, only solvent was added. A constant stream of dry air (20 l/h) was bubbled through the heated oil sample which contained the test solution, and the volatile oxidation products (predominantly short-chain fatty acids such as formic acid or acetic acid) were collected in a receiver containing water. The conductivity of this aqueous solution was continuously measured and documented. The oxidation of (unsaturated) fats proceeds only very slowly for some time and then suddenly increases. The time to the increase is referred to as the induction period (IP).

The following equation was used to calculate the antioxidative index (AOI):

AOI=IP_{(with test solution)}/IP_{(control sample)}.

The results for the experiment at 8° C. in squalene that has been purified over alumina grade N and stabilized with 1 ppm of α-tocopherol are given in table 2:

TABLE 2

| | AOI in squalene at 80° C. |
|---|---|
| Test compound as in | with 0.005% of test |
| example | substance |
| 1 | 69 |
| 2 | 43 |
| 3 | 55 |
| Vitamin C | 0.7 |
| a-Tocopherol | 39 |
| Dibutylhydroxytoluene | 38 |

1. A 3,4-dihydroxymandelic acid alkylamide of the general formula 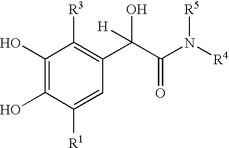
where
R¹, R² and R³, independently of one another, are hydrogen, lower alkyl or groups —O—R⁶ in which R⁶ is hydrogen or lower alkyl, and
R⁴ is hydrogen, an alkyl radical having 1 to 22 carbon atoms or an alkenyl radical having 2 to 22 carbon atoms, and
R⁵ is an alkyl radical having 1 to 22 carbon atoms or an alkenyl radical having 2 to 22 carbon atoms,
including a stereoisomer thereof or mixture thereof.

2. A 3,4-dihydroxymandelic acid alkylamide of the formula 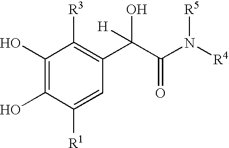
where
R¹, R²and R³, independently of one another, are hydrogen, methyl, tert-butyl, hydroxyl or methoxy, and
R⁴ is hydrogen, and
R⁵ is an alkyl radical having 1 to 18 carbon atoms or an alkenyl radical having 2 to 20 carbon atoms,
including a stereoisomer thereof or mixture thereof.

3. A 3,4-dihydroxymandelic acid alkylamide of the formula 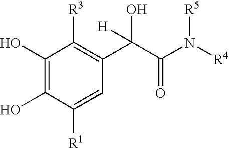
where
R¹, R², R³ and R⁴ are hydrogen, and
R⁵ is an alkyl radical having 1 to 12 carbon atoms or an alkenyl radical having 2 to 12 carbon atoms,
including a stereoisomer thereof or mixture thereof.

4. 2-(3,4-Dihydroxyphenyl)-N-n-hexyl-2-hydroxyacetamide, N-cyclohexyl-2-(3,4-dihydroxyphenyl)-2-hydroxyacetamide and 2-(3,4-dihydroxyphenyl)-N-(2-ethylhexyl)-2-hydroxyacetamide.

5. A cosmetic or pharmaceutical preparation comprising 0.001% by weight to 30% by weight, preferably 0.001 to 20% by weight, particularly preferably 0.01 to 5% by weight, of the 3,4-dihydroxymandelic acid alkylamides as claimed in claims 1 to 4, based on the total weight of the preparation.

6. The use of the 3,4-dihydroxymandelic acid alkylamides as claimed in claims 1 to 4 in cosmetic and pharmaceutical preparations.

7. A food or luxury product comprising 0.001% by weight to 35% by weight, preferably 0.001 to 20% by weight, particularly preferably 0.01 to 0.5% by weight, of the 3,4-dihydroxymandelic acid alkylamides as claimed in claims 1 to 4, based on the total weight of the food or luxury product.

8. The use of the 3,4-dihydroxymandelic acid alkylamides as claimed in claims 1 to 4 in foods or luxury products or for supplementing foods or luxury products.

9. The use of the 3,4-dihydroxymandelic acid alkylamides as claimed in claims 1 to 4 as antioxidants and/or free-radical scavengers.

10. The use of the preparations as claimed in claims 5 to 8 as antioxidants and/or free-radical scavengers.

11. The preparation as claimed in claims 5 to 6 which additionally comprises at least one UVA and/or UVB filter substance.

12. The preparation as claimed in claims 5 to 8 which additionally comprises at least one further antioxidant or a free-radical scavenger.

13. The preparation as claimed in claims 5 to 6 which additionally comprises at least one UVA and/or UVB filter substance and at least one further antioxidant or a free-radical scavenger.

14. A process for the preparation of the 3,4-dihydroxymandelic acid alkylamides of the formula 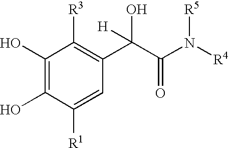
where
R¹, R² and R³, independently of one another, are hydrogen, lower alkyl or groups —O—R⁶ in which R⁶ is hydrogen or lower alkyl, and
R⁴ is hydrogen, an alkyl radical having 1 to 22 carbon atoms or an alkenyl radical having 2 to 22 carbon atoms, and
R⁵ is an alkyl radical having 1 to 22 carbon atoms or an alkenyl radical having 2 to 22 carbon atoms,
including stereoisomers thereof or mixtures thereof,
characterized in that, a 3,4-dihydroxymandelic acid activated in the form of the acid chloride, the acid anhydride or an acid ester, for example of optionally substituted phenols, N-hydroxysuccinimide or N-hydroxybenzotriazole and optionally protected on the phenolic OH groups is reacted with an alkylamine of the general formula HNR⁴R⁵ or an ammonium salt of the general formula (H₂NR⁴R⁵)⁺A⁻, where the radicals R⁴ and R⁵ have the meanings given above and A⁻ is an inorganic or organic anion, for example halide, sulfate, hydrogensulfate or acetate, optionally in the presence of solvents and auxiliary bases, and any protective group which may be present are cleaved off.

15. A process for the preparation of the 3,4-dihydroxymandelic acid alkylamides of the formula 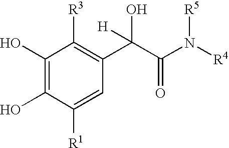
where
R¹, R² and R³, independently of one another, are hydrogen, lower alkyl or groups —O—R⁶ in which R⁶ is hydrogen or lower alkyl, and
R⁴ is hydrogen, an alkyl radical having 1 to 22 carbon atoms or an alkenyl radical having 2 to 22 carbon atoms, and
R⁵ is an alkyl radical having 1 to 22 carbon atoms or an alkenyl radical having 2 to 22 carbon atoms,
including stereoisomers thereof or mixtures thereof, characterized in that
the free 3,4-dihydroxymandelic acids are condensed directly with an alkylamine of the general formula HNR⁴R⁵, where the radicals R⁴ and R⁵ have the meanings given above, with or without solvents with the elimination of water with the aid of a condensing agent, preferably N,N′-dicyclohexylcarbodiimide.

16. The process as claimed in claims 14 and 15, characterized in that the 3,4-dihydroxymandelic acids used are 2-(3,4-dihydroxyphenyl)-2-hydroxyacetic acid, stereoisomers thereof and mixtures thereof.

17. The process as claimed in claims 14 to 16, characterized in that the alkylamines used are hexylamine, 2-ethylhexylamine or cyclohexylamine or the respective ammonium salts.
